(19) United States
(12) Patent Application Publication
(10) Pub. No.: US 2003/0114504 A1
(43) Pub. Date: Jun. 19, 2003

(54) Compositions and methods for the treatment of cancer

(76) Inventors: Kevin R. Webster, Hopkinton, MA (US); Spencer David Kimball, East Windsor, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/228,544

(22) Filed: Aug. 27, 2002

Related U.S. Application Data
(60) Provisional application No. 60/316,369, filed on Aug. 31, 2001.

(51) Int. Cl. A61K 31/427; A61K 31/426

(52) U.S. Cl. 514/369

(57) ABSTRACT

The compounds of the invention are protein kinase inhibitors and are useful in the treatment of proliferative diseases. Compositions and methods are provided for the synergistic treatment of proliferative disorders.

### CROSS REFERENCE TO RELATED APPLICATION

This application claims priority from provisional application serial No. 60/316,369 filed Aug. 31, 2001 which is incorporated herein by reference in its entirety.

### FIELD OF THE INVENTION

This invention relates to the fields of molecular biology and oncology. More specifically, the invention provides compositions and methods for the treatment of proliferative disorders which arise due to aberrant cellular signaling events.

### BACKGROUND OF THE INVENTION

Several literature and patent references are cited throughout the present application. Each of these references is incorporated by reference as though set forth herein in full.

Uncontrolled proliferation is a hallmark of cancer cells. Over the past two decades it has become increasingly clear that during tumorigenesis, molecules that directly control cell cycle progression accumulate defects. These defects can result in the loss of checkpoint control and/or the inappropriate activation of the ‘drivers’ of cell cycle progression, the cyclin-dependent kinases (CDKs). Misregulation of CDK function occurs with high frequency in major solid tumor types (including breast, colon, NSCL, prostate, gastric, bladder and ovarian carcinomas). Therefore, inhibitors of cyclin-dependent kinases and cell cycle progression have the potential to fill a large therapeutic need.

The cyclin-dependent kinases are serine/threonine protein kinases that transduce signals that drive the cell cycle and cell proliferation. CDKs are multisubunit enzymes composed of at least a catalytic subunit and a regulatory (cyclin) subunit (for a review see (1)). To date, 9 CDK and >10 cyclin subunits have been identified which can combine to form in excess of 15 active kinase complexes. In normal cells, many of these enzymes can be categorized as G1, S, or G2/M phase enzymes which perform distinct roles in cell cycle progression. CDKs phosphorylate and modulate the activity of a variety of cellular proteins that include tumor suppressors (e.g. RB, p53), transcription factors (e.g. E2F-DP1, RNA pol II), replication factors (e.g. DNA pol α, replication protein A), and organizational factors which influence cellular and chromatin structures (e.g. histone H1, lamin A, MAP4). CDK activity is regulated through a variety of coordinated mechanisms, which include cell cycle dependent transcription and translation, cell cycle dependent proteolysis, subcellular localization, post-translational modifications and interaction with CDK inhibitor proteins (CKIs). It would be highly desirable to identify agents which modulate the activity of CDKs in methods for treating the aberrant cellular proliferation associated with malignancy.

### SUMMARY OF THE INVENTION

The present invention provides a method for the treatment of anti-proliferative diseases, including cancer, which comprises administering to a mammalian specie in need thereof a synergistically, therapeutically effective amount of: (1) at least one anti-proliferative agent and (2) a Compound of Formula I:

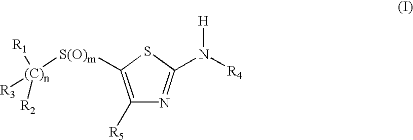

and pharmaceutically acceptable salts thereof. As used in Formula I, and throughout the specification, the symbols have the following meanings:

R₁ and R₂ are independently hydrogen, fluorine or alkyl;

R₃ is aryl or heteroaryl

R₄ is hydrogen, alkyl, cycloalkyl, aryl, cycloalkylalkyl, arylalkyl, heteroaryl, heteroarylalkyl, heterocycloalkyl, heterocycloalkylalkyl; or

CO-alkyl, CO-cycloalkyl, CO-aryl, CO-alkyl-cycloalkyl, CO-alkyl-aryl, CO-heteroaryl, CO-alkyl-heteroaryl, CO-heterocycloalkyl, CO-alkyl-heterocycloalkyl; or

CONH-alkyl, CONH-cycloalkyl, CONH-aryl, CONH-alkyl-cycloalkyl, CONH-alkyl-aryl, CONH-heteroaryl, CONH-alkyl-heteroaryl, CONH-heterocycloalkyl, CONH-alkyl-heterocycloalkyl; or

COO-alkyl, COO-cycloalkyl, COO-aryl, COO-alkyl-cycloalkyl,

COO-alkyl-aryl, COO-heteroaryl, COO-alkyl-heteroaryl, COO-heterocycloalkyl, COO-alkyl-heterocycloalkyl;

or SO₂-alkyl, SO₂-cycloalkyl, SO₂-aryl, SO₂-alkyl-cycloalkyl, SO₂-alkyl-aryl, SO₂-heteroaryl, SO₂-alkyl-heteroaryl, SO₂-heterocycloalkyl, SO₂-alkyl-heterocycloalkyl; or

C(NCN)NH-alkyl, C(NCN)NH-cycloalkyl, C(NCN)NH-aryl, C(NCNNH)-alkyl-cycloalkyl, C(NCN)NH-alkyl-aryl, C(NCN)NH-heteroaryl, C(NCN)NH-alkyl-heteroaryl, C(NCN)NH-heterocycloalkyl, C(NCN)NH-alkyl-heterocylcoalkyl; or

C(NNO,)NH-alkyl, C(NNO,)NH-cycloalkyl, C(NN0,) NH-aryl, C(NNO.)NH-alkyl-cycloalkyl, C(NNO,)NH-alkyl-aryl, C(NNO,)NH-heteroaryl, C(NNO,)NH-alkyl-heteroaryl, C(NNO,)NH-heterocyloalkyl, C(NNO,)NH-alkyl-heterocycloalkyl; or

C(NH)NH-alkyl, C(NH)NH-cycloalkyl, C(NH)NH-aryl, C(NH)NH-alkyl-cycloalkyl, C(NH)NH-alkyl-aryl, C(NH) NH-heteroaryl, C(NH)NH-alkyl-heteroaryl, C(NH)NH-heterocycloalkyl, C(NH)NH-alkyl-heterocycloalkyl; or

C(NH)NHCO-alkyl, C(NH)NHCO-cycloalkyl, C(NH) NHCO-aryl, C(NH)NHCO-alkyl-cycloalkyl, C(NH)NHCO-alkyl-aryl, C(NH)NHCO-heteroaryl, C(NH)NHCO-alkyl-heteroaryl, C(NH)NHCO-heterocylcloalkyl, C(NH)NHCO-alkyl-heterocycloalkyl; or

C(NOR₆)NH-alkyl, C(NOR₆)NH-cycloalkyl, C(NOR₆) NH-aryl, C(NOR₆)NH-alkyl-cycloalkyl, C(NOR₆)NH-alkyl-aryl, C(NOR₆)NH-heteroaryl, C(NOR₆)NH-alkyl-heteroaryl, C(NOR₆)NH-heterocylcoalkyl, C(NOR₆)NH-alkyl-heterocycloalkyl;

R₅ is hydrogen or alkyl;

R₆ is hydrogen, alkyl, cycloalkyl, aryl, cycloalkylakyl, arylalkyl, heteroaryl, heteroarylalkyl, heterocycloalkylalkyl; heterocycloalkyl or

m is an integer of 0 to 2; and

n is an integer of 1 to 3.

The compounds of Formula I are protein kinase inhibitors and are useful in the treatment and prevention of proliferative diseases, for example, cancer, inflammation and arthritis. They may also be useful in the treatment of neurodegenerative diseases such as Alzheimer's disease, cardiovascular diseases, viral diseases and fungal diseases.

The present invention provides for compounds of Formula I, pharmaceutical compositions employing such Compounds and for synergistic methods of using such compounds for the treatment of proliferative disorders.

Listed below are definitions of various terms used to describe the compounds of the instant invention. These definitions apply to the terms as they are used throughout the specification (unless they are otherwise limited in specific instances) either individually or as part of a larger group.

It should be noted that any heteroatom with unsatisfied valances is assumed to have the hydrogen atom to satisfy the valances.

Carboxylate anion refers to a negatively charged group —COO—.

The term “alkyl” or “alk” refers to a monovalent alkane (hydrocarbon) derived radical containing from 1 to 12 carbon atoms unless otherwise defined. An alkyl group is an optionally substituted straight, branched or cyclic saturated hydrocarbon group. When substituted, alkyl groups may be substituted with up to four substituent groups, R as defined, at any available point of attachment. When the alkyl group is said to be substituted with an alkyl group, this is used interchangeably with “branched alkyl group”. Exemplary unsubstituted such groups include methyl, ethyl, propyl, isopropyl, n-butyl, t-butyl, isobutyl, pentyl, hexyl, isohexyl, heptyl , 4,4-dimethylpentyl, octyl, 2,2,4-trimethylpentyl, nonyl, decyl, undecyl, dodecyl, and the like. Exemplary substituents may include but are not limited to one or more of the following groups: halo (such as F, Cl, Br, I), haloalkyl (such as CCl₃, or CF,), alkoxy, alkylthio, hydroxy, carboxy (—COOH), alkyloxycarbonyl (—C(O)R), alkylcarbonyloxy (—OCOR), amino (—NH,), carbamoyl (—NHCOOR— or —OCONHR—), urea (—NHCONHR—) or thiol (SH).

Alkyl groups as defined may also comprise one or more carbon to carbon double bonds or one or more carbon to carbon triple bonds.

The term “alkenyl” refers to a hydrocarbon radical straight, branched or cyclic containing from 2 to 12 carbon atoms and at least one carbon to carbon double bond. The term “alkynyl” refers to a hydrocarbon radical straight, branched or cyclic containing from 2 to 12 carbon atoms and at least one carbon to carbon triple bond.

Cycloalkyl is a specie of alkyl containing from 3 to 15 5s carbon atoms, without alternating or resonating double bonds between carbon atoms. It may contain from 1 to 4 rings. Exemplary unsubstituted such groups include cyclopropyl, cyclobutyl, cyclopentyl, cyclohexyl, adamantyl, etc. Exemplary substituents include one or more of the following groups: halogen, alkyl, alkoxy, alkyl hydroxy, amino, nitro, cyano, thiol and/or alkylthio.

The terms “alkoxy” or “alkylthio”, as used herein, denote an alkyl group as described above bonded through an oxygen linkage (—O—) or a sulfur linkage (—S—), respectively.

The term “alkyloxycarbonyl”, as used herein, denotes an alkoxy group bonded through a carbonyl group. An alkoxy-alkoxycarbonyl radical is represented by the Formula: —C(O)OR, where the R group is a straight or branched C₁₋₆ alkyl group.

The term “alkylcarbonyl” refers to an alkyl group bonded through a carbonyl group.

The term “alkylcarbonyloxy”, as used herein, denotes an alkylcarbonyl group which is bonded through an oxygen linkage.

The term “arylalkyl”, as used herein, denotes an aromatic ring bonded to an alkyl group as described above.

The term “aryl” refers to monocyclic or bicyclic aromatic rings, e.g. phenyl, substituted phenyl and the like, as well as groups which are fused, e.g., napthyl, phenanthrenyl and the like. An aryl group thus contains at least one ring having at least 6 atoms, with up to five such rings being present, containing up to 22 atoms therein, with alternating (resonating) double bonds between adjacent carbon atoms or suitable heteroatoms. Aryl groups may optionally be substituted with one or more groups including, but not limited to halogen, alkyl, alkoxy, hydroxy, carboxy, carbamoyl, alkyloxycarbonyl, nitro, trifluoromethyl, amino, cycloalkyl, cyano, alkyl S(O)ₘ (m=0, 1, 2), or thiol.

The term “heteroaryl” refers to a monocyclic aromatic hydrocarbon group having 5 or 6 ring atoms, or a bicyclic aromatic group having 8 to 10 atoms, containing at least one heteroatom, O, S, or N, in which a carbon or nitrogen atom is the point of attachment, and in which one or two additional carbon atoms is optionally replaced by a heteroatom selected from O or S, and in which from 1 to 3 additional carbon atoms are optionally replaced by nitrogen heteroatoms, said heteroaryl group being optionally substituted as described herein. Exemplary heteroaryl groups include the following: thienyl, furyl, pyrrolyl, pyridinyl, imidazolyl, pyrrolidinyl, piperidinyl, thiazolyl, oxazolyl, triazolyl, pyrazolyl, isoxazolyl, isothiazolyl, pyrazinyl, pyridazinyl, pyrimidinal, triazinylazepinyl, indolyl, isoindolyl, quinolinyl, isoquinolinyl, benzothiazolyl, benzoxazolyl, benzimidazolyl, benzoxadiazolyl, benzofurazanyl and tetrahydropyranyl. Exemplary substituents include one or more of the following: halogen, alkyl, alkoxy, hydroxy, carboxy, carbamoyl, alkyloxycarbonyl, trifluoromethyl, cycloalkyl, nitro, cyano, amino, alkylS(O).sub.m (m=0, 1, 2), or thiol.

The term “heteroarylium” refers to heteroaryl groups bearing a quaternary nitrogen atom and thus a positive charge.

The term “heterocycloalkyl” refers to a cycloalkyl group (nonaromatic) in which one of the carbon atoms in the ring is replaced by a heteroatom selected from O, S or N, and in which up to three additional carbon atoms may be replaced by said heteroatoms.

The term “quaternary nitrogen” refers to a tetravalent positively charged nitrogen atom including, e.g. the positively charged nitrogen in a tetraalkylammonium group (e.g. tetramethylammonium, N-methylpyridinium), the positively charged nitrogen in protonated ammonium species (e.g. trimethylhydroammonium, N-hydropyridinium), the positively charged nitrogen in amine N-oxides (e.g. N-methyl-morpholine-N-oxide, pyridine —N-oxide), and the positively charged nitrogen in an N-amino-ammonium group (e.g. N-aminopyridinium).

The term “heteroatom” means O, S or N, selected on an independent basis.

The term “halogen” or “halo” refers to chlorine, bromine, fluorine or iodine.

When a functional group is termed “protected”, this means that the group is in modified form to preclude undesired side reactions at the protected site. Suitable protecting groups for the compounds of the present invention will be recognized from the present application taking into account the level of skill in the art, and with reference to standard textbooks, such as Greene, T. W. et al., Protective Groups in Organic Synthesis, 2d Ed., John Wiley & Sons, Inc., N.Y. (1991).

Suitable examples of salts of the compounds according to the invention with inorganic or organic acids are hydrochloride, hydrobromide, sulfate, tartrate and phosphate. Salts of the compounds of the invention encompass solvates, racemates and all stereoisomeric forms thereof, including enantiomers and diastereomers (for example, D-tartrate and L-tartrate salts). Salts which are unsuitable for pharmaceutical uses but which can be employed, for example, for the isolation or purification of free compounds I or their pharmaceutically acceptable salts, are also included.

An example of a compound of Formula I is Formula II shown below:

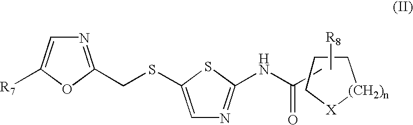

and enantiomers, diastereomers and pharmaceutically acceptable salts thereof wherein

R₇ is alkyl;

R₈ is hydrogen or alkyl;

X is NR₉ or CHNR₉R₁₀;

R₉ and R₁₀ are each independently hydrogen, alkyl, substituted alkyl, cycloalkyl or substituted cycloalkyl; and

n is 0, 1, 2 or 3.

All stereoisomers of the compounds of the instant invention are contemplated, either in admixture or in pure or substantially pure form. The definition of the compounds according to the invention embraces all possible stereoisomers and their mixtures. It very particularly embraces the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates by conventional methods, such as, for example, salt formation with an optically active acid followed by crystallization.

It should be understood that solvates (e.g., hydrates) of the compounds of Formula I are also within the scope of the present invention. Methods of solvation are generally known in the art.

The compounds of Formula I are particularly useful as potent, protein kinase inhibitors and are useful in methods for the treatment of proliferative diseases, for example, cancer, inflammation and arthritis. They may also be useful in the treatment of Alzheimer's disease, chemotherapy-induced alopecia, and cardiovascular disease.

Suitable anti-proliferative agents for use in the synergistic methods of the invention, include, without limitation, alkylating agents (including, without limitation, nitrogen mustards, ethylenimine derivatives, alkyl sulfonates, nitrosoureas and triazenes): Uracil mustard, Chlormethine, Cyclophosphamide, Cytoxan® Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide; antimetabolites (including, without limitation, folic acid antagonists, pyrimidine analogs, purine analogs and adenosine deaminase inhibitors), Methotrexate, 5-Fluorouracil, Floxuridine, Cytarabine, 6-Mercaptopurine, 6-Thioguanine, Fludarabine phosphate, Pentostatine, and Gemcitabine; natural products and their derivatives (for example, vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, Ara-C, paclitaxel (paclitaxel is commercially available as Taxol®), Mithramycin, Deoxyco-formycin, Mitomycin-C, L-Asparaginase, Interferons (especially IFN-a), Etoposide, and Teniposide; navelbene, CPT-11, anastrazole, letrazole, capecitabine, reloxafine, cyclophosphamide, ifosamide, and droloxafine, epothilone A, epothilone B, epothilone C, epothilone D, desoxyepothilone A, desoxyepothilone B, [1S-1R*,3R*(E),7R*,10S*, 11R*,12R*,16S*]]-7-11-dihydroxy-8,8,10,12,16-pentamethyl-3-[1-methyl-2-(2-methyl-4-thiazolyl)ethenyl]-4-aza-17 oxabicyclo [14.1.0]heptadecane-5,9-dione (disclosed in WO 99/02514), [1S-[1R*,3R*(E),7R*,10S*,11R*,12R*,16S*]]-3-[2-[2-(aminomethyl)-4-thiazolyl]-1-methylethenyl]-7,11-dihydroxy-8,8,10,12,16-pentamethyl-4-17-dioxabicyclo[14.1.0]-heptadecane-5,9-dione (disclosed in U.S. Pat. No. 6,260,694, issued Jul. 17, 2001 and derivatives thereof; other microtubule-disruptor agents, and radiation.

The present invention further provides a pharmaceutical composition for the synergistic treatment of cancer which comprises at least one anti-proliferative agent, and a compound of Formula I, and a pharmaceutically acceptable carrier.

In one embodiment of the invention the antiproliferative agent is administered before the administration of a compound of Formula I. In another embodiment of the invention, the anti-proliferative agent is administered simultaneously with the compound of Formula I. In yet another embodiment, the compound of Formula I is administered before the anti-proliferative agent.

### BRIEF DESCRIPTION OF THE DRAWINGS

FIG. 1. The anatomy of a cell cycle checkpoint.

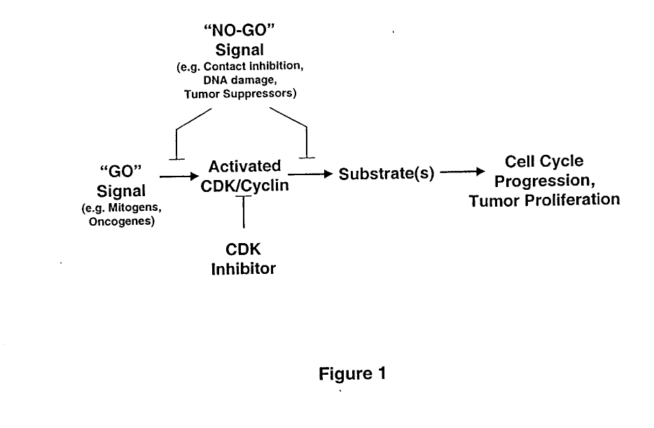

FIG. 2. A schematic of restriction point control. CDK2 is a key regulator of the restriction ® point, a cell cycle checkpoint governing the passage from G1 to S phase of the cell cycle.

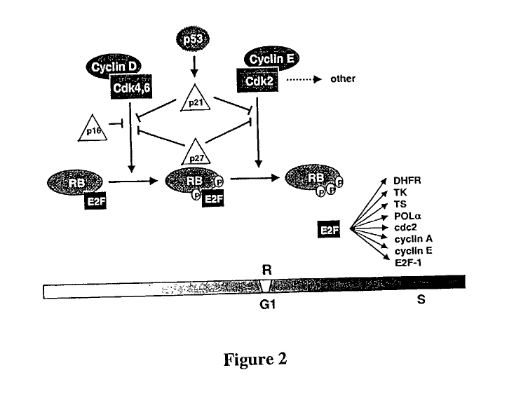

FIG. 3. PARP-cleavage is induced following exposure to Compound 1. A2780S cells were treated for 0, 1, 2, 4, 6, and 24 hours with 20, 100 or 200 nM compound. Protein extracts were then examined by western blot using an anti-PARP antibody (Clonetech). The arrow signifies caspase cleaved PARP protein fragment.

FIG. 4. Comparative antitumor activity of Compound 1, 5-FU and paclitaxel versus the Colo205 human colon carcinoma model. Compound was administered at the indicated doses on treatment regimens of ip,q1dx8, iv,q7dx3, and iv,q2dx5, respectively. Each datum point represents the median tumor weight of 8 mice. Horizontal bar indicates tumor growth delay equivalent to 1 LCK.

FIG. 5. Synergistic interaction of Compound 1 in combination with a farnesyl transferase inhibitor, Compound 2, and the DNA crosslinker, cisplatin, in an in vitro clonogenic assay versus A2780s ovarian carcinoma cells. Various concentrations of either Compound 2 or cisplatin were combined with 1.5 μM Compound 1. The black triangles represent Compound 2 or Cisplatin alone, the red circles represent the combined cytotoxicity of Compound 1 with either Compound 2 or Cisplatin and the blue line represents the line of multiplicity. The line of multiplicity depicts the level of cytotoxicity if the two combined agents yield additive cytotoxicity and is the product of the surviving fractions of each agent given independently. The sequence and time of drug exposure are the following. A) Cells were treated during times 0-4 hours with Compound 1 followed by treatment with Compound 2 at times 4-24 hours or Cisplatin at times 24-28 hours. B) Cells were treated during times 0-20 hours with Compound 2 or during time 0-4 hours with Cisplatin followed by treatment with Compound 1 at times 4-24 hours or 24-28 hours for Compound 2 and cisplatin respectively. C) Cells were treated with both agents simultaneously during times 0-4 hours. In all cases colony formation was scored on day 10-14.

### DETAILED DESCRIPTION OF THE INVENTION

In accordance with the present invention, methods for the scheduled administration of inhibitors of cyclin-dependent kinases in synergistic combination(s) with at least one additional anti-neoplastic or anti-proliferative agent for the treatment and prevention of proliferative diseases are provided.

An exemplary compound of Formula I, Compound 1 is a rationally designed inhibitor of CDK2. The potency and selectivity profile of this compound was optimized to yield maximal anti-tumor effects while maintaining a clear therapeutic window. Compound 1 inhibits CDK2 with an IC₅₀=48 nM. This compound is 10-fold and 100-fold less potent against the highly related protein kinases CDK1 and CDK4 respectively. Compound 1 demonstrated remarkable selectivity (>500-fold) against 15 unrelated Serine/Threonine and Tyrosine protein kinases. Compound 1 is a potent and broadly active inhibitor of tumor cell proliferation in vitro. Treatment results in abrupt inhibition of cell cycle progression followed by an apoptotic response. Clonogenic assays indicate that 8 hours of drug exposure is sufficient to elicit a maximal anti-proliferative response in vitro. The activity of Compound 1 is additive or synergistic when combined with key front-line cancer therapeutics in vitro. Compound 1 exhibits broad spectrum anti-tumor activity in multiple murine and human tumor models in vivo. These include the P388 mouse leukemia, Cyclin E transgenic mouse breast carcinoma, A2780 human ovarian carcinoma, Colo205 human colorectal carcinoma and A431 human squamous cell carcinoma. Compound 1 demonstrated curative efficacy at multiple dose levels in the A2780 human tumor xenograft when dosed IP on a qdx8 schedule. Activity is dose and schedule dependent with qdx8=qdx14>>>q2dx5=q4dx3. Compound 1 demonstrated modest efficacy in the A2780 xenograft model when dosed orally on a qdx8 schedule. In addition, treatment of A2780 tumor bearing mice with a single 24 hour continuous infusion of Compound 1 results in curative efficacy at the maximally tolerated dose (MTD).

Thus, in a preferred embodiment, the chemotherapeutic method of the invention comprises the administration of a CDK2 inhibitor of Formula I in combination with other anti-cancer agents. The CDK inhibitors disclosed herein, when used in combination with at least one other anti-cancer agent(s) demonstrate superior cytotoxic activity.

In a preferred embodiment of the invention a compound of Formula I is administered in conjunction with at least one anti-neoplastic agent.

As used herein, the phrase “anti-neoplastic agent” is synonymous with “chemotherapeutic agent” and/or “anti-proliferative agent” and refers to compounds that prevent cancer, or hyperproliferative cells from multiplying. Anti-proliferative agents prevent cancer cells from multiplying by: (1) interfering with the cell's ability to replicate DNA and (2) inducing cell death and/or apoptosis in the cancer cells.

Classes of compounds that may be used as anti-proliferative cytotoxic agents include the following:

Alkylating agents (including, without limitation, nitrogen mustards, ethylenimine derivatives, alkyl sulfonates, nitrosoureas and triazenes): Uracil mustard, Chlormethine, Cyclophosphamide, Cytoxan®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide.

Antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine.

Natural products and their derivatives (for example, vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, Ara-C, paclitaxel (paclitaxel is commercially available as Taxol®), Mithramycin, Deoxyco-formycin, epothilone A, epothilone B, epothilone C, epothilone D, desoxyepothilone A, desoxyepothilone B, [1S-1R*,3R*(E),7R*,10S*,11R*,12R*,16S*]]-7-11-dihydroxy-8,8,10,12,16-pentamethyl-3-[1-methyl-2-(2-methyl-4-thiazolyl)ethenyl]-4-aza-17 oxabicyclo [14.1.0]heptadecane-5,9-dione (disclosed in WO 99/02514), [1S-[1R*,3R*(E),7R*,10S*,11R*,12R*,16S*]]-3-[2-[2-(aminomethyl)-4-thiazolyl]-1-methylethenyl]-7,11-dihydroxy-8,8,10,12,16-pentamethyl-4-17-dioxabicyclo[14.1.0]-heptadecane-5,9-dione (disclosed in U.S. Pat. No. 6,260,694, issued Jul. 17, 2001 and derivatives thereof, Mitomycin-C, L-Asparaginase, Interferons (especially IFN-a), Etoposide, and Teniposide.

Other anti-proliferative cytotoxic agents are navelbene, CPT-11, anastrazole, letrazole, capecitabine, reloxafine, cyclophosphamide, ifosamide, and droloxafine.

The phrase “radiation therapy” includes, but is not limited to, x-rays or gamma rays which are delivered from either an externally applied source such as a beam or by implantation of small radioactive sources

Microtubule affecting agents interfere with cellular mitosis and are well known in the art for their anti-proliferative cytotoxic activity. Microtubule affecting agents useful in the invention include, but are not limited to, allocolchicine (NSC 406042), Halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol®, NSC 125973), Taxol® derivatives (e.g., derivatives (e.g., NSC 608832), thiocolchicine NSC 361792), trityl cysteine (NSC 83265), vinblastine sulfate (NSC 49842), vincristine sulfate (NSC 67574), natural and synthetic epothilones including but not limited to epothilone A, epothilone B, epothilone C, epothilone D, desoxyepothilone A, desoxyepothilone B, [1S-[1R*,3R*(E),7R*,10S*,11R*,12R*,16S*]]-7-11-dihydroxy-8,8,10,12,16-pentamethyl-3-[1-methyl-2-(2-methyl-4-thiazolyl)ethenyl]-4-aza-17 oxabicyclo [14.1.0]heptadecane-5,9-dione (disclosed in WO 99/02514), [1S-[1R*,3R*(E),7R*,10S*,11R*,12R*,16S*]]-3-[2-[2-(aminomethyl)-4-thiazolyl]-1-methylethenyl]-7,11-dihydroxy-8,8,10,12,16-pentamethyl-4-17-dioxabicyclo[14.1.0]-heptadecane-5,9-dione (disclosed in U.S. Pat. No. 6,260,694, issued Jul. 17, 2001) and derivatives thereof; and other microtubule-disruptor agents. Additional antineoplastic agents include, discodermolide (see Service, (1996) Science, 274:2009) estramustine, nocodazole, MAP4, and the like. Examples of such agents are also described in the scientific and patent literature, see, e.g., Bulinski (1997) J. Cell Sci. 110:3055 3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem 271:29807-29812.

In cases where it is desirable to render aberrantly proliferative cells quiescent in conjunction with or prior to treatment with the chemotherapeutic methods of the invention, hormones and steroids (including synthetic analogs): 17a-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolone, chlorotrianisene, Hydroxyprogesterone, Aminoglutethimide, Estramustine, Medroxyprogesteroneacetate, Leuprolide, Flutamide, Toremifene, Zoladex can also be administered to the patient.

Also suitable for use in the combination chemotherapeutic methods of the invention are antiangiogenics such as matrix metalloproteinase inhibitors, and other VEGF inhibitors, such as anti-VEGF antibodies and small molecules such as ZD6474 and SU6668 are also included. Anti- Her2 antibodies from Genetech may also be utilized. A suitable EGFR inhibitor is EKB-569 (an irreversible inhibitor). Also included are Imclone antibody C225 immunospecific for the EGFR, and src inhibitors.

Also suitable for use as an antiproliferative cytostatic agent is Casodex® which renders androgen-dependent carcinomas non-proliferative. Yet another example of a cytostatic agent is the antiestrogen Tamoxifen which inhibits the proliferation or growth of estrogen dependent breast cancer. Inhibitors of the transduction of cellular proliferative signals are cytostatic agents. Examples are epidermal growth factor inhibitors, Her-2 inhibitors, MEK-1 kinase inhibitors, MAPK kinase inhibitors, PI3 inhibitors, Src kinase inhibitors, and PDGF inhibitors.

As mentioned, certain anti-proliferative agents are anti-angiogenic and antivascular agents and, by interrupting blood flow to solid tumors, render cancer cells quiescent by depriving them of nutrition. Castration, which also renders androgen dependent carcinomas non-proliferative, may also be utilized. Starvation by means other than surgical disruption of blood flow is another example of a cytostatic agent. A particularly preferred class of antivascular cytostatic agents is the combretastatins. Other exemplary cytostatic agents include MET kinase inhibitors, MAP kinase inhibitors, inhibitors of non-receptor and receptor tyrosine kinases, inhibitors of integrin signaling, and inhibitors of insulin-like growth factor receptors.

Thus, the present invention provides methods for the synergistic treatment of a variety of cancers, including, but not limited to, the following:

carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma);

hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma;

hematopoietic tumors of myeloid lineage including acute and chronic myelogenous leukemias, myelodysplastic syndrome, myeloid leukemia, and promyelocytic leukemia;

tumors of the central and peripheral nervous system including astrocytoma, neuroblastoma, glioma, and schwannomas;

tumors of mesenchymal origin including fibrosarcoma, rhabdomyoscarcoma, and osteosarcoma; and

other tumors including melanoma, xenoderma pigmentosum, keratoactanthoma, seminoma, thyroid follicular cancer, and teratocarcinoma.

Most preferably, the invention is used to treat accelerated or metastatic cancers of the bladder, pancreatic cancer, prostate cancer, non-small cell lung cancer, colorectal cancer, ovarian and breast cancer.

In a preferred embodiment of this invention, a method is provided for the synergistic treatment of cancerous tumors. Advantageously, the synergistic method of this invention reduces the development of tumors, reduces tumor burden, or produces tumor regression in a mammalian host.

Methods for the safe and effective administration of most of these chemotherapeutic agents are known to those skilled in the art. In addition, their administration is described in the standard literature.

For example, the administration of many of the chemotherapeutic agents is described in the “Physicians' Desk Reference” (PDR), e.g., 1996 edition (Medical Economics Company, Montvale, N.J. 07645-1742, USA); the disclosure of which is incorporated herein by reference thereto.

Methods for the synthesis of the compounds of Formula I are provided in U.S. Pat. No. 6,040,321, the entire disclosure of which is incorporated herein by reference.

The compounds of Formula I are useful in various pharmaceutically acceptable salt forms. The term “pharmaceutically acceptable salt” refers to those salt forms which would be apparent to the pharmaceutical chemist, i.e., those which, while maintaining therapeutic effect, provide the desired pharmacokinetic properties, palatability, absorption, distribution, metabolism or excretion. Other factors, more practical in nature, which are also important in the selection, are cost of the raw materials, ease of crystallization, yield, stability, hygroscopicity and flowability of the resulting bulk drug. Conveniently, pharmaceutical compositions may be prepared from the active ingredients or their pharmaceutically acceptable salts in combination with pharmaceutically acceptable carriers.

Pharmaceutically acceptable salts of the Formula I compounds which are suitable for use in the methods and compositions of the present invention include, but are not limited to, salts formed with a variety of organic and inorganic acids such as hydrogen chloride, hydroxymethane sulfonic acid, hydrogen bromide, methanesulfonic acid, sulfuric acid, acetic acid, trifluoroacetic acid, maleic acid, benzenesulfonic acid, toluenesulfonic acid, sulfamic acid, glycolic acid, stearic acid, lactic acid, malic acid, pamoic acid, sulfanilic acid, 2-acetoxybenzoic acid, fumaric acid, toluenesulfonic acid, methanesulfonic acid, ethanedisulfonic acid, oxalic acid, isethonic acid, and include various other pharmaceutically acceptable salts, such as, e.g., nitrates, phosphates, borates, tartrates, citrates, succinates, benzoates, ascorbates, salicylates, and the like. Cations such as quaternary ammonium ions are contemplated as pharmaceutically acceptable counterions for anionic moieties.

Preferred salts of Formula I compounds include tartrate salts, hydrochloride salts, methanesulfonic acid salts and trifluoroacetic acid salts. In addition, pharmaceutically acceptable salts of the Formula I compounds may be formed with alkali metals such as sodium, potassium and lithium; alkaline earth metals such as calcium and magnesium; organic bases such as dicyclohexylamine, tributylamine, and pyridine; and amino acids such as arginine, lysine and the like.

The pharmaceutically acceptable salts of the present invention can be synthesized by conventional chemical methods. Generally, the salts are prepared by reacting the free base or acid with stoichiometric amounts or with an excess of the desired salt-forming inorganic or organic acid or base, in a suitable solvent or solvent combination.

The present invention also encompasses a pharmaceutical composition useful in the treatment of cancer, comprising the administration of a therapeutically effective amount of the combinations of this invention, with or without pharmaceutically acceptable carriers or diluents. The synergistic pharmaceutical compositions of this invention comprise an anti-proliferative agent or agents, a Formula I compound, and a pharmaceutically acceptable carrier. The methods entail the use of a neoplastic agent in combination with a Formula I compound. The compositions of the present invention may further comprise one or more pharmaceutically acceptable additional ingredient(s) such as alum, stabilizers, antimicrobial agents, buffers, coloring agents, flavoring agents, adjuvants, and the like. The antineoplastic agents, Formula I compounds and compositions of the present invention may be administered orally or parenterally including the intravenous, intramuscular, intraperitoneal, subcutaneous, rectal and topical routes of administration.

For oral use, the antineoplastic agents, Formula I compounds and compositions of this invention may be administered, for example, in the form of tablets or capsules, powders, dispersible granules, or cachets, or as aqueous solutions or suspensions. In the case of tablets for oral use, carriers which are commonly used include lactose, corn starch, magnesium carbonate, talc, and sugar, and lubricating agents such as magnesium stearate are commonly added. For oral administration in capsule form, useful carriers include lactose, corn starch, magnesium carbonate, talc, and sugar. When aqueous suspensions are used for oral administration, emulsifying and/or suspending agents are commonly added.

In addition, sweetening and/or flavoring agents may be added to the oral compositions. For intramuscular, intraperitoneal, subcutaneous and intravenous use, sterile solutions of the active ingredient(s) are usually employed, and the pH of the solutions should be suitably adjusted and buffered. For intravenous use, the total concentration of the solute(s) should be controlled in order to render the preparation isotonic.

For preparing suppositories according to the invention, a low melting wax such as a mixture of fatty acid glycerides or cocoa butter is first melted, and the active ingredient is dispersed homogeneously in the wax, for example by stirring. The molten homogeneous mixture is then poured into conveniently sized molds and allowed to cool and thereby solidify.

Liquid preparations include solutions, suspensions and emulsions. Such preparations are exemplified by water or water/propylene glycol solutions for parenteral injection. Liquid preparations may also include solutions for intranasal administration.

Aerosol preparations suitable for inhalation may include solutions and solids in powder form, which may be in combination with a pharmaceutically acceptable carrier, such as an inert compressed gas.

Also included are solid preparations which are intended for conversion, shortly before use, to liquid preparations for either oral or parenteral administration. Such liquid forms include solutions, suspensions and emulsions.

The compounds of Formula I, as well as the anti-neoplastic agents, described herein may also be delivered transdermally. The transdermal compositions can take the form of creams, lotions, aerosols and/or emulsions and can be included in a transdermal patch of the matrix or reservoir type as are conventional in the art for this purpose.

The combinations of the present invention may also be used in conjunction with other well known therapies that are selected for their particular usefulness against the condition that is being treated.

If formulated as a fixed dose, the active ingredients of the combination compositions of this invention are employed within the dosage ranges described below. Alternatively, the anti-neoplastic, and Formula I compounds may be administered separately in the dosage ranges described below. In a preferred embodiment of the present invention, the antineoplastic agent is administered in the dosage range described below simultaneously, before, or after administration of the Formula I compound.

Table I sets forth preferred chemotherapeutic combinations and exemplary dosages for use in the methods of the present invention. Where “Compound of Formula I” appears, any of the variations of Formula I set forth herein, including the salt form shown above are contemplated for use in the chemotherapeutic combinations. Preferably, Compound 1 is employed.

TABLE 1

| CHEMOTHERAPEUTIC | DOSAGE | |
|---|---|---|
| COMBINATION | mg/m² (per dose) | |
| Compound of Formula I | 1.0-100 | mg/m2 |
| + Cisplatin | 5-150 | mg/m2 |
| Compound of Formula 1 | 1.0-100 | mg/m2 |
| + Compound 2 | 25-500 | mg/m2 |
| Compound of Formula I | 1.0-100 | mg/m2 |
| + Carboplatin | 5-1000 | mg/m2 |
| Compound of Formula I | 1.0-100 | mg/m2 |
| + Radiation | 200-8000 | cGy |
| Compound of Formula I | 1.0-100 | mg/m2 |
| + CPT-11 | 5-400 | mg/m2 |
| Compound of Formula I | 1.0-100 | mg/m2 |
| + Paclitaxel | 40-250 | mg/m2 |
| Compound of Formula I | 1.0-100 | mg/m2 |
| + Paclitaxel | 40-250 | mg/m2 |
| + Carboplatin | 5-1000 | mg/m2 |
| Compound of Formula I | 1.0-100 | mg/m2 |
| + 5FU and optionally | 5-5000 | mg/m2 |
| + Leucovorin | 5-1000 | mg/m2 |
| Compound of Formula I | 1.0-100 | mg/m2 |
| + Epothilone | 1-500 | mg/m2 |
| Compound of Formula I | 1.0-100 | mg/m2 |
| + Gemcitabine | 100-3000 | mg/m2 |
| Compound of Formula I | 1.0-100 | mg/m2 |
| + UFT and optionally | 50-800 | mg/m2 |
| + leucovorin | 5-1000 | mg/m2 |
| Compound of Formula I | 1.0-100 | mg/m2 |
| + Gemcitabine | 100-3000 | mg/m2 |
| + Cisplatin | 5-150 | mg/m2 |
| Compound of Formula I | 1.0-100 | mg/m2 |
| + UFT | 50-800 | mg/m2 |
| + Leucovorin | 5-1000 | mg/m2 |
| Compound of Formula I | 1.0-100 | mg/m2 |
| + Cisplatin | 5-150 | mg/m2 |
| + paclitaxel | 40-250 | mg/m2 |
| Compound of Formula I | 1.0-100 | mg/m2 |
| + Cisplatin | 5-150 | mg/m2 |
| + 5FU | 5-5000 | mg/m2 |
| Compound of Formula I | 1.0-100 | mg/m2 |
| + Oxaliplatin | 5-200 | mg/m2 |
| + CPT-11 | 4-400 | mg/m2 |
| Compound of Formula I | 1.0-100 | mg/m2 |
| + 5FU | 5-5000 | mg/m2 |
| + CPT-11 and optionally | 4-400 | mg/m2 |
| + leucovorin | 5-1000 | mg/m2 |
| Compound of Formula I | 1.0-100 | mg/m2 |
| + 5FU | 5-5000 | mg/m2 |
| + radiation | 200-8000 | cGy |
| Compound of Formula I | 1.0-100 | mg/m2 |
| + radiation | 200-8000 | cGy |
| + 5FU | 5-5000 | mg/m2 |
| + Cisplatin | 5-150 | mg/m2 |
| Compound of Formula I | 1.0-100 | mg/m2 |
| + Oxaliplatin | 5-200 | mg/m2 |
| + 5FU and optionally | 5-5000 | mg/m2 |
| + Leucovorin | 5-1000 | mg/m2 |
| Compound of Formula I | 1.0-100 | mg/m2 |
| + paclitaxel | 40-250 | mg/m2 |
| + CPT-11 | 4-400 | mg/m2 |
| Compound of Formula I | 1.0-100 | mg/m2 |
| + paclitaxel | 40-250 | mg/m2 |
| + 5-FU | 5-5000 | mg/m2 |
| Compound of Formula I | 1.0-100 | mg/m2 |
| + UFT | 50-800 | mg/m2 |
| + CPT-11 and optionally | 4-400 | mg/m2 |
| + leucovorin | 5-1000 | mg/m2 |

In the above Table I, “5FU” denotes 5-fluorouracil, “Leucovorin” can be employed as leucovorin calcium, “UFT” is a 1:4 molar ratio of tegafur:uracil, and “Epothilone” is preferably a compound described in WO 99/02514 or WO 00/50423, both incorporated by reference herein in their entirety.

While Table I provides exemplary dosage ranges of the Formula I compounds and certain anticancer agents of the invention, when Formulating the pharmaceutical compositions of the invention the clinician may utilize preferred dosages as warranted by the condition of the patient being treated. For example, Compound 1 may preferably administered at 3-60 mg/m2 every 3 weeks. Compound 2, may preferably be administered at a dosage ranging from 25-500 mg/m2 every three weeks for as long as treatment is required. Preferred dosages for cisplatin are 75-120 mg/m2 administered every three weeks. Preferred dosages for carboplatin are within the range of 200-600 mg/m2 or an AUC of 0.5-8 mg/ml×min; most preferred is an AUC of 4-6 mg/ml×min. When the method employed utilizes radiation, preferred dosages are within the range of 200-6000 cGY. Preferred dosages for CPT-11 are within 100-125 mg/m2, once a week. Preferred dosages for paclitaxel are 130-225 mg/m2 every 21 days. Preferred dosages for gemcitabine are within the range of 80-1500 mg/m2 administered weekly. Preferably UFT is used within a range of 300-400 mg/m2 per day when combined with leucovorin administration. Preferred dosages for leucovorin are 10-600 mg/m2 administered weekly.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

Certain cancers can be treated effectively with compounds of Formula I and a plurality of anticancer agents. Such triple and quadruple combinations can provide greater efficacy. When used in such triple and quadruple combinations the dosages set forth above can be utilized. Other such combinations in the above Table I can therefore include “Compound 1” in combination with (1) mitoxantrone+prednisone; (2) doxorubicin+carboplatin; or (3 herceptin+tamoxifen. 5-FU can be replaced by UFT in any of the above combinations.

When employing the methods or compositions of the present invention, other agents used in the modulation of tumor growth or metastasis in a clinical setting, such as antiemetics, can also be administered as desired.

The present invention encompasses a method for the synergistic treatment of cancer wherein a neoplastic agent and a Formula I compound are administered simultaneously or sequentially. Thus, while a pharmaceutical Formulation comprising antineoplastic agent(s) and a Formula I compound may be advantageous for administering the combination for one particular treatment, prior administration of the anti-neoplastic agent(s) may be advantageous in another treatment. It is also understood that the instant combination of antineoplastic agent(s) and Formula I compound may be used in conjunction with other methods of treating cancer (preferably cancerous tumors) including, but not limited to, radiation therapy and surgery. It is further understood that a cytostatic or quiescent agent, if any, may be administered sequentially or simultaneously with any or all of the other synergistic therapies.

The combinations of the instant invention may also be co-administered with other well known therapeutic agents that are selected for their particular usefulness against the condition that is being treated. Combinations of the instant invention may alternatively be used sequentially with known pharmaceutically acceptable agent(s) when a multiple combination Formulation is inappropriate.

The chemotherapeutic agent(s) and/or radiation therapy can be administered according to therapeutic protocols well known in the art. It will be apparent to those skilled in the art that the administration of the chemotherapeutic agent(s) and/or radiation therapy can be varied depending on the disease being treated and the known effects of the chemotherapeutic agent(s) and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., antineoplastic agent(s) or radiation) on the patient, and in view of the observed responses of the disease to the administered therapeutic agents.

In the methods of this invention, a compound of Formula I is administered simultaneously or sequentially with an anti-proliferative agent and/or radiation. Thus, it is not necessary that the chemotherapeutic agent(s) and compound of Formula I, or the radiation and the compound of Formula I, be administered simultaneously or essentially simultaneously. The advantage of a simultaneous or essentially simultaneous administration is well within the determination of the skilled clinician.

Also, in general, the compound of Formula I, and chemotherapeutic agent(s) do not have to be administered in the same pharmaceutical composition, and may, because of different physical and chemical characteristics, have to be administered by different routes. For example, the compound of Formula I may be administered orally to generate and maintain good blood levels thereof, while the chemotherapeutic agent(s) may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of compound of Formula I and anti-proliferative cytotoxic agent(s) or radiation will depend upon the diagnosis of the attending physicians and their judgment of the condition of the patient and the appropriate treatment protocol.

If the compound of Formula I and the anti-neoplastic agent(s) and/or radiation are not administered simultaneously or essentially simultaneously, then the initial order of administration of the compound of Formula I, and the chemotherapeutic agent(s) and/or radiation, may be varied. Thus, for example, the compound of Formula I may be administered first followed by the administration of the antiproliferative agent(s) and/or radiation; or the antiproliferative agent(s) and/or radiation may be administered first followed by the administration of the compound of Formula I. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the patient. For example, the anti-neoplastic agent(s) and/or radiation may be administered initially, especially if a cytotoxic agent is employed. The treatment is then continued with the administration of the compound of Formula I and optionally followed by administration of a cytostatic agent, if desired, until the treatment protocol is complete.

Thus, in accordance with experience and knowledge, the practicing physician can modify each protocol for the administration of a component (therapeutic agent-i.e., compound of Formula I, anti-neoplastic agent(s), or radiation) of the treatment according to the individual patient's needs, as the treatment proceeds.

The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

In order to facilitate a further understanding of the invention, the following examples are presented primarily for the purpose of illustrating more specific details thereof. The scope of the invention should not be deemed limited by the examples, but to encompass the entire subject matter defined by the claims.

Experimental Protocol—Compounds:

The following designations are used to identify the test compounds throughout the examples:

Compound 1: N-[5-[[[5-(1,1-Dimethylethyl)-2-oxazolyl]methyl]thio]-2-thiazolyl]-4-piperidinecarboxamide.

Compound 2: (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt.

The following materials and methods are provided to facilitate the practice of the methods of the invention.

In vitro Studies

Compounds.

All compounds were synthesized by the medicinal Chemistry group at Bristol-Myers Squibb Pharmaceutical Research Institute. Compounds were solubilized in 100% DMSO at a concentration of 10 mM for all experiments. Compound dilutions were made into respective growth media.

Cell Culture.

Cell lines were maintained in RPMI-1640 plus 10% fetal bovine serum.

CDK1/cyclin B1 Kinase Assay.

Kinase reactions consisted of 100 ng of baculovirus expressed GST- CDK1/cyclin B1 complex, 1 μg histone H1 (Boehringer Mannheim, Indianapolis, Ind.), 0.2 μCi ³³P γ-ATP, 25 μM ATP in 50 μl kinase buffer (50 mM Tris, pH 8.0, 10 mM MgCl₂, 1 mM EGTA, 0.5 mM DTT). Reactions were incubated for 45 minutes at 30° C. and stopped by the addition of cold trichloroacetic acid (TCA) to a final concentration 15%. TCA precipitates were collected onto GF/C unifilter plates (Packard Instrument Co., Meriden, Conn.) using a Filtermate universal harvester (Packard Instrument Co., Meriden, Conn.) and the filters were quantitated using a TopCount 96-well liquid scintillation counter (Packard Instrument Co., Meriden, Conn.). Dose response curves were generated to determine the concentration required to inhibit 50% of kinase activity (IC₅₀). Compounds were dissolved at 10 mM in DMSO and evaluated at six concentrations, each in triplicate. The final concentration of DMSO in the assay equaled 2%. IC₅₀ values were derived by non-linear regression analysis and have a coefficient of variance (SD/mean, n=6)=16%.

CDK 2/cyclin E Kinase Assay.

Kinase reactions consisted of 5 ng of baculovirus expressed GST- CDK2/cyclin E complex, 0.5 μg GST-RB fusion protein (amino acids 776-928 of retinoblastoma protein), 0.2 μCi ³³P γ-ATP, 25 μM ATP in 50 μl kinase buffer (50 mM Hepes, pH 8.0, 10 mM MgCl₂, 1 mM EGTA, 2 mM DTT). Reactions were incubated for 45 minutes at 30° C. and stopped by the addition of cold trichloroacetic acid (TCA) to a final concentration 15%. TCA precipitates were collected onto GF/C unifilter plates (Packard Instrument Co., Meriden, Conn.) using a Filtermate universal harvester (Packard Instrument Co., Meriden, Conn.) and the filters were quantitated using a TopCount 96-well liquid scintillation counter (Packard Instrument Co., Meriden, Conn.). Dose response curves were generated to determine the concentration required inhibiting 50% of kinase activity (IC₅₀). Compounds were dissolved at 10 mM in DMSO and evaluated at six concentrations, each in triplicate. The final concentration of DMSO in the assay equaled 2%. IC₅₀ values were derived by non-linear regression analysis and have a coefficient of variance (SD/mean, n=6)=14%.

CDK4/cyclin D1 Kinase Assay.

Kinase reactions consisted of 150 ng of baculovirus expressed GST- CDK4, 280 ng of Stag-cyclin D1, 0.5 μg GST-RB fusion protein (amino acids 776-928 of retinoblastoma protein), 0.2 μCi ³³P γ-ATP, 25 μM ATP in 50 μl kinase buffer (50 mM Hepes, pH 8.0, 10 mM MgCl₂, 1 mM EGTA, 2 mM DTT). Reactions were incubated for 1 hour at 30° C. and stopped by the addition of cold trichloroacetic acid (TCA) to a final concentration 15%. TCA precipitates were collected onto GF/C unifilter plates (Packard Instrument Co., Meriden, Conn.) using a Filtermate universal harvester (Packard Instrument Co., Meriden, Conn.) and the filters were quantitated using a TopCount 96-well liquid scintillation counter (Packard Instrument Co., Meriden, Conn.). Dose response curves were generated to determine the concentration required inhibiting 50% of kinase activity (IC₅₀). Compounds were dissolved at 10 mM in DMSO and evaluated at six concentrations, each in triplicate. The final concentration of DMSO in the assay equaled 2%. IC₅₀ values were derived by non-linear regression analysis and have a coeficient of variance (SD/mean, n=6)=18%.

Cell Cycle Analysis.

Log phase A2780s cells were plated overnight in 6 well plates. Cells were treated with different concentrations of Compound 1 for varying times. Cells were harvested by trypsinization followed by centrifugation. Cell pellets were then resuspended by vortexing in 1 ml 80% methanol and fixed overnight at −20° C. Cells were recovered by centrifugation and washed two times with 1 ml of PBS. Cells were resuspended in 1 ml PBS, 2% FBS, 0.25% Triton X-100 and incubated at 4° C. for 10 minutes. Cells were again recovered by centrifugation and resuspended in 50 μl PBS, 2% FBS, 0.1% Triton X-100. Anti-Phospho-Threonine Proline antibody (IgM, New England Biolabs #9391S) was added and the cells were incubated for 30 minutes at 4° C. Cells were washed with PBS, 2% FBS, 0.1% Triton X-100 and resuspended in 50 μl PBS, 2% FBS, 0.1% Triton X-100. FITC-anti-Mouse antibody (Pharmingen #12064D) was added and incubated for 30 minutes at 4° C. in the dark. Cells were washed with PBS, 2% FBS, 0.1% Triton X-100 and resuspended in Propidium Iodide/RNase in PBS (10 μg/ml PI, 100 μg/ml RNase (DNase free)) and incubated at 37° C. for 30 minutes in the dark. Samples were analyzed using a flow cytometer.

Western Blot Analysis.

Compound treated A2780S cells were harvested at approximately 70% confluence and total protein was prepared by lysing the cells in RIPA [50 mM Tris (pH8), 150 mM NaCl, 1% NP-40, 0.5% Na-deoxycolate, 0.1% SDS, 0.1% Na3VO4, 0.1 mM NaF, 10 mM β-glycerophosphate, plus Complete® protease inhibitors (Boehringer Mannhiem)] buffer. Cell pellets were resuspended at a density of<2×10⁷ cells/ml and incubated for 20 minutes on ice followed by a high speed 14,000 rpm centrifugation. The protein supernatant was then removed from the debris and protein content was quantitated using the Micro-BCA assay (Pierce). Treated extracts (25 microgram/lane) were then separated using a 10% SDS-polyacrylamide gel (10.5×14 cm). Proteins were then transferred from the gel to PVDF-membrane (Millipore) by exposure to 0.8 Amp/cm² in a semi-dry blotting apparatus (Hoeffer). PVDF protein blots were then blocked with 5% non-fat milk in TTBS (0.1% Tween 20 in Tris-buffered saline). Blots were then probed with primary antibody in 5% non-fat milk in TTBS for 1-2 hours, followed by three washes with TTBS. An HRP-conjugated secondary antibody was then incubated with the blots in TTBS for 30 minutes. The blots were then washed three times with TTBS and developed with ECL-plus western blotting detection system (Amersham).

Clonogenic Growth Assay and Drug Combination Studies.

Colony growth inhibition was measured for A2780 ovarian carcinoma cells using a standard clonogenic assay. Briefly, 200 cells/well were seeded into 6-well tissue culture plates (Falcon, Franklin Lakes, N.J.) and allowed to attach for 18 hours. Assay medium consisted of RPMI-1640 plus 10% fetal bovine serum. Cells were then treated in duplicate with a six concentration dose-response curve. The maximum concentration of DMSO never exceeded 0.25%. For combination studies cells were exposed to the compound 1 for indicated time which was then removed and the cells were washed with 2 volumes of PBS. The normal growth medium was then replaced or the cells were exposed to compound 2. After the final compound exposure the cells were washed with 2 volumes of PBS and the normal growth medium was then replaced. Colonies were fed with fresh media every third day. Colony number was scored on day 10-14 using a Optimax imaging station. The compound concentration required to inhibit 50% or 90% of colony formation (TC₅₀ or IC₉₀, respectively) was determined by non-linear regression analysis. The coefficient of variance (SD/mean, n=3)=30%. The effects of combination treatment was evaluated using the multiplicity method described by Stephens and Steel (2). This method assumes a simple linear isobologram, meaning that each individual agent demonstrates a linear dose response curve. This assumption allows for the generation of a theoretical curve, termed the line of multiplicity, that represents the expected additive response.

In vivo Antitumor Testing

Drug Administration.

Compound 1 was first dissolved in a mixture of Cremophor®/ethanol (50:50). Final dilution to the required dosage strength was made with water so that the dosing solutions contained Cremophor®/ethanol/water at a ratio of 10:10:80, respectively. Paclitaxel was dissolved in a 50/50 mixture of ethanol and Cremophor® and stored at 4° C.; final dilution of paclitaxel was obtained immediately before drug administration with NaCl 0.9%. 5-FU was dissolved in normal saline (NaCl 0.9%). Flavopiridol was dissolved in Cremophor®/ethanol/water at a ratio of 10:10:80. The volume of all compounds injected was 0.01 ml/g of mouse weight.

Animals.

All rodents were obtained from Harlan Sprague Dawley Co. (Indianpolis, Ind.), and maintained in an ammonia-free environment in a defined and pathogen-free colony. The animal care program of Bristol-Myers Squibb Pharmaceutical Research Institute is fully accredited by the American Association for Accreditation of Laboratory Animal Care (AAALAC).

Solid Tumor Xenografts in Nude Mice.

The following tumors were used: A2780 human ovarian carcinoma, Br-cycE murine breast carcinoma, A431 human squamous cell carcinoma and Colo 205 colorectal carcinoma.

All solid tumors were maintained in Balb/c nu/nu nude mice. Tumors were propagated as subcutaneous transplants using tumor fragments obtained from donor mice. All tumor implants for efficacy testing were subcutaneous (sc).

The required number of animals needed to detect a meaningful response were grouped at the start of the experiment and each was given a subcutaneous implant of a tumor fragment (˜50 mg) with a 13-gauge trocar. For treatment of early-stage tumors, the animals were again grouped before distribution to the various treatment and control groups. For treatment of animals with advanced-stage disease, tumors were allowed to grow to the pre-determined size window (animals with tumors outside the range were excluded) and animals were evenly distributed to various treatment and control groups. Treatment of each animal was based on individual body weight. Treated animals were checked daily for treatment related toxicity/mortality. Each group of animals was weighed before the initiation of treatment (Wt1) and then again following the last treatment dose (Wt2). The difference in body weight (Wt2-Wt1) provides a measure of treatment-related toxicity.

Tumor response was determined by measurement of tumors with a caliper twice a week, until the tumors reach a predetermined “target” size of 1 g. Tumor weights (mg) were estimated from the Formula:

Tumor weight=(length×width²)÷2

Antitumor activity was evaluated at the maximum tolerated dose (MTD) which is defined as the dose level immediately below which excessive toxicity (i.e. more than one death) occurred. The MTD was frequently equivalent to optimal dose (OD). When death occured, the day of death was recorded. Treated mice dying prior to having their tumors reach target size were considered to have died from drug toxicity. No control mice died bearing tumors less than target size. Treatment groups with more than one death caused by drug toxicity were considered to have had excessively toxic treatments and their data were not included in the evaluation of a compound's antitumor efficacy.

Tumor response end-point was expressed in terms of tumor growth delay (T-C value), defined as the difference in time (days) required for the treated tumors (T) to reach a predetermined target size compared to those of the control group ®.

To estimate tumor cell kill, the tumor volume doubling time (TVDT) was first calculated with the Formula:

TVDT=Median time (days) for control tumors to reach target size−Median time (days) for control tumors to reach half the target size

And,

Log cell kill=T−C÷(3.32×TVDT)

Statistical evaluations of data were performed using Gehan's generalized Wilcoxon test.

### EXAMPLE 1

In vitro Studies

The activity of Compound 1 was evaluated against human recombinant CDK2 and a panel of protein kinases in vitro (3). Compound 1 potently inhibited the phosphorylation of RB protein by CDK2 in vitro with an IC₅₀ of 48 nM (Table 2). The mechanism of inhibition is through direct competition with the ATP substrate. Compound 1 was less potent against other members of the cyclin-dependent kinase family with an IC₅₀ of 480 and 925 nM against CDK1 and CDK4, respectively.

TABLE 2
Potencies of kinase inhibition by Compound 1
and flavopiridol in vitro.

| | Flavopiridol | Compound 1 |
|---|---|---|
| Protein Kinase | IC₅₀ (nM) | IC₅₀ (nM) |
| CDK1/cyclin B | 30 | 480 |
| CDK2/cyclin E | 170 | 48 |
| CDK4/cyclin D1 | 100 | 925 |

The effect of Compound 1 on cell cycle progression at 8 and 24 hours post-treatment is shown in Table 3. The drug concentration used in this experiment was equivalent to an IC₉₀ for a 72 hour treatment (170 nM). Drug exposure of 8 hours was sufficient to alter the normal cell cycle distribution. These effects are even more pronounced in the 24 hour drug treatment sample. Compound 1 causes a dramatic decrease in both the S- and M- phase cell populations and a dramatic increase in the sub-G1 or apoptotic cells.

TABLE 3
Compound 1 alters cell cycle
distribution and induces apoptosis.

| Cell Cycle | | | | | |
|---|---|---|---|---|---|
| Profile, A2780S | % G1 | % S | % G2 | % M | % Apo* |
| Control | 53 | 20 | 16.5 | 2.5 | 3 |
| 8 hr. exposure, | 56 | 15 | 22 | 1 | 6 |
| 170 nM | | | | | |
| 24 hr. exposure, | 45 | 8 | 9.7 | 0.3 | 32 ± 12 |
| 170 nM | | | | | |

*Degree of apoptosis is scored by DNA staining of sub-G1 cells.

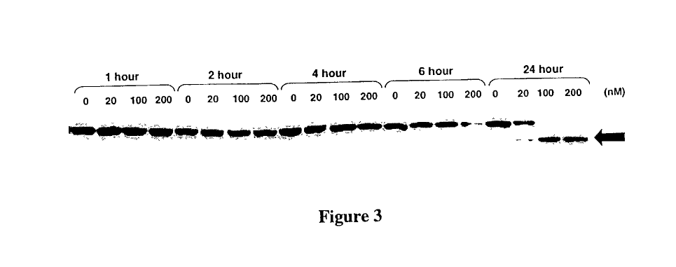

The mechanism of apoptosis was further confirmed and defined by monitoring the activation of caspases following treatment with Compound 1. PARP cleavage is an accepted marker for the activation of caspase cascades (e.g. caspase 3). Following exposure of A2780s cells to Compound 1, protein extracts were made and PARP status was inspected by western blot (FIG. 3). It is clear that the 110 kD native PARP protein is being digested by as early as 6 hours of drug exposure. This is signaled by the appearance of the cleaved 85 kD PARP protein fragment. Extensive cleavage (i.e. caspase activation) is apparent in the 24 hour time point, consistent with the appearance of sub-G1 cells noted above. These observations confirm that Compound 1 treatment results in an apoptotic or programmed cell death.

In vivo Efficacy

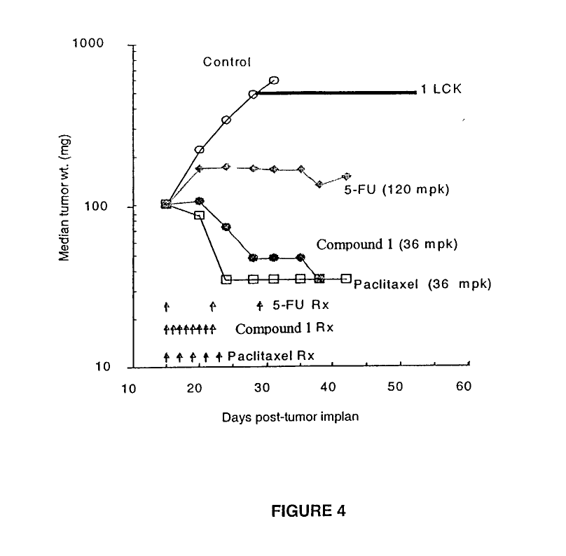

The in vivo efficacy of Compound 1 has been evaluated in 5 preclinical in vivo cancer models, including ip/ip P388 murine leukemia, sc A2780 human ovarian carcinoma, Br-cycE murine mammary carcinoma, A431 human squamous cell carcinoma, and Colo205 human colon carcinoma (6). Compound 1 was compared head to head with flavopiridol in each of these models. In addition, the route of administration, schedule dependency and minimum effective exposure was determined for Compound 1 (6). The data obtained against the colo205 rectal cancer line is shown in FIG. 4.

Colo205 human colon carcinoma.

Compound 1 was evaluated in head-to-head comparison with two reference agents (5-FU and paclitaxel) against the Colo205 human colon carcinoma. Compound 1 demonstrated marked antitumor activity producing>2.0 LCK and tumor regression at the MTD of 36 mg/kg, IP, QD×8. Paclitaxel, administered at its MTD and optimal schedule (36 mg/kg, Q2%×5, IV) produced antitumor activity comparable to Compound 1 (FIG. 4). However, 5-FU was considerably less active and failed to achieve tumor regression in this model.

Combination chemotherapy in vitro.

The success of the CDK2 inhibitor, Compound 1, is dependent not only on its antitumor activity as a single agent but also its ability to combine successfully with other antineoplastic drugs. A cell cycle inhibitor could be used to synchronize a tumor cell population, thus priming it for subsequent destruction by a phase specific cytotoxic agent. In fact, this has already been demonstrated in vitro for the early CDK inhibitors, flavopiridol and olomoucine. Researchers have demonstrated that flavopiridol can potentiate the action of a variety of agents including, cisplatin, mitomycin C, paclitaxel, cytarabine, topotecan, doxorubicin, etoposide, and 5-fluorouracil in vitro. These findings suggest that cell cycle specific agents could be used to improve the therapeutic window for some existing chemotherapies or sensitize normally resistant tumors.

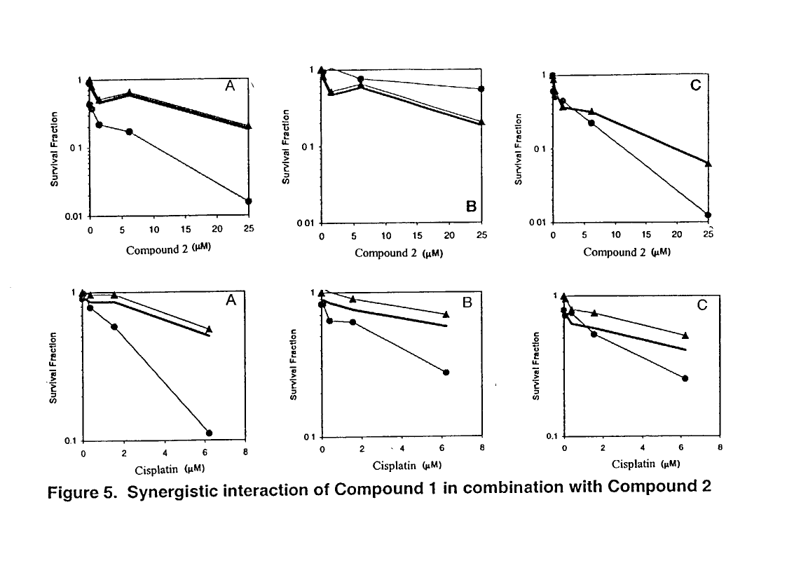

Colony formation assays have been used to test Compound 1 in combination with several anticancer agents in vitro (5). The data were analyzed using the method of multiplicity which assumes a simple linear isobologram, meaning that each individual agent demonstrates a linear dose response curve. This assumption allows for the generation of a theoretical curve, termed the line of multiplicity, that represents the expected additive response. This analysis has shown that the mode of interaction between Compound 1 and other agents in vitro is drug-, sequence- and dose-dependent. Synergy was clearly observed when Compound 1 was combined with either Compound 2 or Cisplatin (Table 4, FIG. 5). This is evident from the shift in the dose response curve, for the combined agents, to the left of the theoretical line of multiplicity. These interactions are sequence dependent. In both instances treatment with Compound 1 prior to exposure to Compound 2 or Cisplatin yields a synergistic interaction (FIG. 5, panel A). Alternate sequences resulted in weaker synergy or an additive interaction (i.e. the survival curve for the combined agents concurs with the theoretical line of multiplicity) with the exception of Compound 2 followed by Compound 1 (FIG. 5, panel B). This combination was antagonistic (i.e., shifted to the right of the theoretical line of multiplicity) under these conditions. Combination of Compound 1 with Paclitaxel, Gemcitabine or Doxorubicin is additive under these conditions regardless of sequence.

TABLE 4
The effect of sequence of drug exposure on the
cytotoxic interaction between Compound 1 and five other
antineoplastic agents in the A2780s human ovarian
carcinoma cell line.

| Combination | |
|---|---|
| Sequence | Mode of Interaction |
| + Compound 2 (farnesyl transferase | |
| inhibitor) | |
| Compound 1 followed by Compound 2 | Synergy |
| Compound 2 followed by Compound 1 | Antagonistic |
| Simultaneous | Weak synergy |
| + Cisplatin (Cisplatin) | |
| Compound 1 followed by Cisplatin | Synergy |
| Cisplatin followed by Compound 1 | Weak synergy |
| Simultaneous | Additive |
| + (Paclitaxel) | |
| Compound 1 followed by Paclitaxel | Additive |
| Paclitaxel followed by Compound 1 | Additive |
| Simultaneous | Not Done |
| + (Doxorubicin) | |
| Compound 1 followed by Doxorubicin | Additive |
| Doxorubicin followed by Compound 1 | Additive |
| Simultaneous | Additive |
| + (Gemcitabine) | |
| Compound 1 followed by Gemcitabine | Additive |
| Gemcitabine followed by Compound 1 | Weak synergy |
| Simultaneous | Additive |

In summary, Compound 1 synergizes with Compound 2 and Cisplatin in colony formation assays in vitro. This activity is sequence dependent. Combination of Compound 1 with Paclitaxel, Gemcitabine and Doxorubicin provides an additive response under the conditions evaluated in this study.

The present invention is not limited to the embodiments specifically described above, but is capable of variation and modification without departure from the scope of the appended claims.

What is claimed is:

1. A method for the treatment of proliferative diseases, including cancer, which comprises administering to a mammalian specie in need thereof a therapeutically effective amount of (1) at least one anti-proliferative agent(s) and (2) a compound of Formula I: 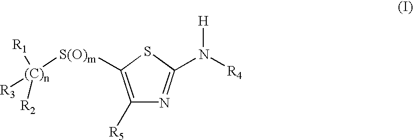
and pharmaceutically acceptable salts, wherein:
R₁ and R₂ are, independently, hydrogen, fluorine or alkyl;
R₃ is aryl or heteroaryl
R₄ is hydrogen, alkyl, cycloalkyl, aryl, cycloalkylalkyl, arylalkyl, heteroaryl, heteroarylalkyl, heterocycloalkyl, heterocycloalkylalkyl; or
CO-alkyl, CO-cycloalkyl, CO-aryl, CO-alkyl-cycloalkyl, CO-alkyl-aryl, CO-heteroaryl, CO-alkyl-heteroaryl, CO-heterocycloalkyl, CO-alkyl-heterocycloalkyl; or
CONH-alkyl, CONH-cycloalkyl, CONH-aryl, CONH-alkyl-cycloalkyl, CONH-alkyl-aryl, CONH-heteroaryl, CONH-alkyl-heteroaryl, CONH-heterocycloalkyl, CONH-alkyl-heterocycloalkyl; or
COO-alkyl, COO-cycloalkyl, COO-aryl, COO-alkyl-cycloalkyl, COO-alkyl-aryl, COO-heteroaryl, COO-alkyl-heteroaryl, COO-heterocycloalkyl, COO-alkyl-heterocycloalkyl;
or SO₂-alkyl, SO₂-cycloalkyl, SO₂-aryl, SO₂-alkyl-cycloalkyl, SO₂-alkyl-aryl, SO₂-heteroaryl, SO₂-alkyl,- heteroaryl, SO₂-heterocycloalkyl, SO₂-alkyl-heterocycloalkyl; or
C(NCN)NH-alkyl, C(NCN)NH-cycloalkyl, C(NCN)NH-aryl, C(NCNNH)-alkyl-cycloalkyl, C(NCN)NH-alkyl-aryl, C(NCN)NH-heteroaryl, C(NCN)NH-alkyl-heteroaryl, C(NCN)NH-heterocycloalkyl, C(NCN)NH-alkyl-heterocylcoalkyl; or
C(NNO,)NH-alkyl, C(NNO,)NH-cycloalkyl, C(NN0,) NH-aryl, C(NNO.)NH-alkyl-cycloalkyl, C(NNO,)NH-alkyl-aryl, C(NNO,)NH-heteroaryl, C(NNO,)NH-alkyl-heteroaryl, C(NNO,)NH-heterocyloalkyl, C(NNO,)NH-alkyl-heterocycloalkyl; or
C(NH)NH-alkyl, C(NH)NH-cycloalkyl, C(NH)NH-aryl, C(NH)NH-alkyl-cycloalkyl, C(NH)NH-alkyl-aryl, C(NH) NH-heteroaryl, C(NH)NH-alkyl-heteroaryl, C(NH)NH-heterocycloalkyl, C(NH)NH-alkyl-heterocycloalkyl; or
C(NH)NHCO-alkyl, C(NH)NHCO-cycloalkyl, C(NH) NHCO-aryl, C(NH)NHCO-alkyl-cycloalkyl, C(NH)NHCO-alkyl-aryl, C(NH)NHCO-heteroaryl, C(NH)NHCO-alkyl-heteroaryl, C(NH)NHCO-heterocylcloalkyl, C(NH)NHCO-alkyl-heterocycloalkyl; or
C(NOR₆)NH-alkyl, C(NOR₆)NH-cycloalkyl, C(NOR₆) NH-aryl, C(NOR₆)NH-alkyl-cycloalkyl, C(NOR₆)NH-alkyl-aryl, C(NOR₆)NH-heteroaryl, C(NOR₆)NH-alkyl-heteroaryl, C(NOR₆)NH-heterocylcoalkyl, C(NOR₆)NH-alkyl-heterocycloalkyl;
R₅ is hydrogen or alkyl;
R₆ is hydrogen, alkyl, cycloalkyl, aryl, cycloalkylakyl, arylalkyl, heteroaryl, heteroarylalkyl, heterocycloalkylalkyl; heterocycloalkyl or
m is an integer of 0 to 2; and
n is an integer of 1 to 3.

2. The method according to claim 1, wherein the Formula I compound is 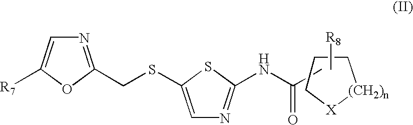
and enantiomers, diastereomers and pharmaceutically acceptable salts thereof, wherein:
R₇ is alkyl;
R₈ is hydrogen or alkyl;
X is NR₉ or CHNR₉R₁₀;
R₉ and R₁₀ are each independently hydrogen, alkyl, substituted alkyl, cycloalkyl or substituted cycloalkyl; and
n is 0, 1, 2 or 3.

3. The method according to claim 2, wherein the Formula I compound is N-[5-[[[5-(1,1-Dimethylethyl)-2-oxazolyl]methyl]thio]-2-thiazolyl]-4-piperidinecarboxamide or a pharmaceutically acceptable salt thereof.

4. The method according to claim 3 wherein said pharmaceutically acceptable salt is a tartrate salt.

5. The method according to claim 1 wherein the antiproliferative agent is administered prior to administration of the Formula I compound.

6. The method according to claim 1 wherein the antiproliferative agent is administered following administration of the Formula I compound.

7. The method according to claim 1 wherein the antiproliferative agent is administered simultaneously with the Formula 1 compound.

8. The method according to claim 1 for the treatment of cancerous solid tumors.

9. The method according to claim 1 for the treatment of refractory tumors.

10. The method according to claim 1 wherein the anti-proliferative agent is selected from the group consisting of a microtubule-stabilizing agent, a microtubule-disruptor agent, an alkylating agent, an anti-metabolite, epidophyllotoxin, an antineoplastic enzyme, a topoisomerase inhibitor, procarbazine, mitoxantrone, radiation and a platinum coordination complex.

11. The method according to claim 1 wherein the anti-proliferative agent is selected from the group consisting of an anthracycline drug, a vinca drug, a mitomycin, a bleomycin, a cytotoxic nucleoside, a taxane, an epothilone, discodermolide, a pteridine drug, a diynene, an aromatase inhibitor and a podophyllotoxin.

12. The method according to claim 1, wherein said method comprises the administration of a compound of Formula I and the anti-proliferative agent is Compound 2.

13. The method according to claim 2, wherein the antiproliferative agent is Compound 2.

14. The method according to claim 3, wherein the antiproliferative agent is Compound 2.

15. The method according to claim 1, wherein said method comprises the administration of a compound of Formula I and the anti-proliferative agent is Cisplatin.

16. The method according to claim 2, wherein the antiproliferative agent is Cisplatin.

17. The method according to claim 3, wherein the antiproliferative agent is Cisplatin.

18. The method according to claim 1, wherein said method comprises the administration of a compound of Formula I and the anti-proliferative agent is Carboplatin.

19. The method according to claim 2, wherein the antiproliferative agent is Carboplatin.

20. The method according to claim 3, wherein the antiproliferative agent is Carboplatin.

21. The method according to claim 1, wherein said method comprises the administration of a compound of Formula I and the anti-proliferative agent is Gemcitabine.

22. The method according to claim 2, wherein the antiproliferative agent is Gemcitabine.

23. The method according to claim 3, wherein the antiproliferative agent is Gemcitabine.

24. The method according to claim 10, wherein said compound of Formula I is selected from the group consisting of:
N-[5-[[[5-(1,1-dimethylethyl)-2-oxazolyl]methyl]thio]-2-thiazolyl]-4-piperidinecarboxamide;
(±)-N-[5-[[[5-(1,1-dimethylethyl)-2-oxazolyl]methyl]thio]-2-thiazolyl]-3-piperidinecarboxamide;
(±)-1-(2,3-dihydroxypropyl)-N-[5-[[[5-(1,1-dimethylethyl)-2-oxazolyl]methyl]thio]-2-thiazolyl]-4-piperidinecarboxamide;
N-[5-[[[5-(1,1-dimethylethyl)-2-oxazolyl]methyl]thio]-2-thiazolyl]-1-(1-methylethyl)-4-piperidinecarboxamide;
1-cyclopropyl-N-[5-[[[5-(1,1-dimethylethyl)-2-oxazolyl]methyl]thio]-2-thiazolyl]-4-piperidinecarboxamide;
N-[5-[[[5-(1,1-dimethylethyl)-2-oxazolyl]methyl]thio]-2-thiazolyl]-1-(2-hydroxyethyl)-4-piperidinecarboxamide;
(R)-N-[5-[[[5-(1,1-dimethylethyl)-2-oxazolyl]methyl]thio]-2-thiazolyl]-3-piperidinecarboxamide;
(S)-N-[5-[[[5-(1,1-dimethylethyl)-2-oxazolyl]methyl]thio]-2-thiazolyl]-3-piperidinecarboxamide;
cis-4-amino-N-[5-[[[5-(1,1-dimethylethyl)-2-oxazolyl]methyl]thio]-2-thiazolyl]cyclohexylcarboxamide; and
trans-4-amino-N-[5-[[[5-(1,1-dimethylethyl)-2-oxazolyl]methyl]thio]-2-thiazolyl]cyclohexylcarboxamide; and
pharmaceutically acceptable salts thereof.

25. The method according to claim 11, wherein said compound of Formula I is selected from the group consisting of N-[5-[[[5-(1,1-dimethylethyl)-2-oxazolyl]methyl]thio]-2-thiazolyl]-4-piperidinecarboxamide;
(±)-N-[5-[[[5-(1,1-dimethylethyl)-2-oxazolyl]methyl]thio]-2-thiazolyl]-3-piperidinecarboxamide;
(±)-1-(2,3-dihydroxypropyl)-N-[5-[[[5-(1,1-dimethylethyl)-2-oxazolyl]methyl]thio]-2-thiazolyl]-4-piperidinecarboxamide;
N-[5-[[[5-(1,1-dimethylethyl)-2-oxazolyl]methyl]thio]-2-thiazolyl]-1-(1-methylethyl)-4-piperidinecarboxamide;
1-cyclopropyl-N-[5-[[[5-(1,1-dimethylethyl)-2-oxazolyl]methyl]thio]-2-thiazolyl]-4-piperidinecarboxamide;
N-[5-[[[5-(1,1-dimethylethyl)-2-oxazolyl]methyl]thio]-2-thiazolyl]-1-(2-hydroxyethyl)-4-piperidinecarboxamide;
(R)-N-[5-[[[5-(1,1-dimethylethyl)-2-oxazolyl]methyl]thio]-2-thiazolyl]-3-piperidinecarboxamide;
(S)-N-[5-[[[5-(1,1-dimethylethyl)-2-oxazolyl]methyl]thio]-2-thiazolyl]-3-piperidinecarboxamide;
cis-4-amino-N-[5-[[[5-(1,1-dimethylethyl)-2-oxazolyl]methyl]thio]-2-thiazolyl]cyclohexylcarboxamide; and
trans-4-amino-N-[5-[[[5-(1,1-dimethylethyl)-2-oxazolyl]methyl]thio]-2-thiazolyl]cyclohexylcarboxamide; and
pharmaceutically acceptable salts thereof.

26. A pharmaceutical composition for the treatment of cancer which comprises at least one anti-proliferative agent and a compound of Formula I as described in claim 1, and a pharmaceutically acceptable carrier.

27. A pharmaceutical composition for the treatment of cancer which comprises at least one anti-proliferative agent and a compound of Formula I as described in claim 2, and a pharmaceutically acceptable carrier.

28. A pharmaceutical composition for the treatment of cancer which comprises at least one anti-proliferative agent and a compound of Formula I as described in claim 3, and a pharmaceutically acceptable carrier.

29. The pharmaceutical composition according to claim 26 for the synergistic treatment of cancerous solid tumors.

30. The pharmaceutical composition according to claim 27 for the synergistic treatment of cancerous solid tumors.

31. The pharmaceutical composition according to claim 28 for the synergistic treatment of cancerous solid tumors.

32. The pharmaceutical composition according to claim 26 for the treatment of refractory tumors.

33. The pharmaceutical composition according to claim 27 for the treatment of refractory tumors.

34. The pharmaceutical composition according to claim 28 for the treatment of refractory tumors.

35. The pharmaceutical composition according to claim 26 wherein the antiproliferative agent is one or more agent selected from the group consisting of a microtubule-stabilizing agent, a microtubule-disruptor agent, an alkylating agent, an anti-metabolite, epidophyllotoxin, an antineoplastic enzyme, a topoisomerase inhibitor, procarbazine, mitoxantrone, a platinum coordination complex, an anthracycline drug, a vinca drug, a mitomycin, a bleomycin, a cytotoxic nucleoside, a taxane, compound 2, an epothilone, discodermolide, a pteridine drug, a diynene, an aromatase inhibitor and a podophyllotoxin.

36. The pharmaceutical composition according to claim 27 wherein the antiproliferative agent is one or more agent selected from the group consisting of a microtubule-stabilizing agent, a microtubule-disruptor agent, an alkylating agent, an anti-metabolite, epidophyllotoxin, an antineoplastic enzyme, a topoisomerase inhibitor, procarbazine, mitoxantrone, a platinum coordination complex, an anthracycline drug, a vinca drug, a mitomycin, a bleomycin, a cytotoxic nucleoside, a taxane, compound 2, an epothilone, discodermolide, a pteridine drug, a diynene, an aromatase inhibitor and a podophyllotoxin.

37. The pharmaceutical composition according to claim 28 wherein the antiproliferative agent is one or more agent selected from the group consisting of a microtubule-stabilizing agent, a microtubule-disruptor agent, an alkylating agent, an anti-metabolite, epidophyllotoxin, an antineoplastic enzyme, a topoisomerase inhibitor, procarbazine, mitoxantrone, a platinum coordination complex, an anthracycline drug, a vinca drug, a mitomycin, a bleomycin, a cytotoxic nucleoside, a taxane, compound 2, an epothilone, discodermolide, a pteridine drug, a diynene, an aromatase inhibitor and a podophyllotoxin.

38. The pharmaceutical composition according to claim 26 wherein the compound of Formula I is selected from the group consisting of N-[5-[[[5-(1,1-dimethylethyl)-2-oxazolyl]methyl]thio]-2-thiazolyl]-4-piperidinecarboxamide;
(±)-N-[5-[[[5-(1,1-dimethylethyl)-2-oxazolyl]methyl]thio]-2-thiazolyl]-3-piperidinecarboxamide;
(±)-1-(2,3-dihydroxypropyl)-N-[5-[[[5-(1,1-dimethylethyl)-2-oxazolyl]methyl]thio]-2-thiazolyl]-4-piperidinecarboxamide;
N-[5-[[[5-(1,1-dimethylethyl)-2-oxazolyl]methyl]thio]-2-thiazolyl]-1-(1-methylethyl)-4-piperidinecarboxamide;
1-cyclopropyl-N-[5-[[[5-(1,1-dimethylethyl)-2-oxazolyl]methyl]thio]-2-thiazolyl]-4-piperidinecarboxamide;
N-[5-[[[5-(1,1-dimethylethyl)-2-oxazolyl]methyl]thio]-2-thiazolyl]-1-(2-hydroxyethyl)-4-piperidinecarboxamide;
(R)-N-[5-[[[5-(1,1-dimethylethyl)-2-oxazolyl]methyl]thio]-2-thiazolyl]-3-piperidinecarboxamide;
(S)-N-[5-[[[5-(1,1-dimethylethyl)-2-oxazolyl]methyl]thio]-2-thiazolyl]-3-piperidinecarboxamide;
cis-4-amino-N-[5-[[[5-(1,1-dimethylethyl)-2-oxazolyl]methyl]thio]-2-thiazolyl]cyclohexylcarboxamide; and
trans-4-amino-N-[5-[[[5-(1,1-dimethylethyl)-2-oxazolyl]methyl]thio]-2-thiazolyl]cyclohexylcarboxamide; and
pharmaceutically acceptable salts thereof.

39. The pharmaceutical composition according to claim 26 wherein the pharmaceutically acceptable salt is selected from the group consisting of the tartrate salt, hydrochloride salt, the methanesulfonic acid salt and the trifluoroacetic acid salt.

40. The pharmaceutical composition according to claim 27 wherein the pharmaceutically acceptable salt is selected from the group consisting of the tartrate salt, hydrochloride salt, the methanesulfonic acid salt and the trifluoroacetic acid salt.

41. The pharmaceutical composition according to claim 28 wherein the pharmaceutically acceptable salt is selected from the group consisting of the tartrate salt, hydrochloride salt, the methanesulfonic acid salt and the trifluoroacetic acid salt.

42. The pharmaceutical composition according to claim 26 wherein the Formula I compound is N-[5-[[[5-(1,1-Dimethylethyl)-2-oxazolyl]methyl]thio]-2-thiazolyl]-4-piperidinecarboxamide or a pharmaceutically acceptable salt thereof and the anti-proliferative agent is Compound 2.

43. The pharmaceutical composition according to claim 26 wherein the antiproliferative agent is Cisplatin and the Formula I compound is N-[5-[[[5-(1,1-Dimethylethyl)-2-oxazolyl]methyl]thio]-2-thiazolyl]-4-piperidinecarboxamide or a pharmaceutically acceptable salt thereof.

44. The pharmaceutical composition according to claim 26 wherein the antiproliferative agent is gemcitabine and the compound of Formula I is N-[5-[[[5-(1,1-Dimethylethyl)-2-oxazolyl]methyl]thio]-2-thiazolyl]-4-piperidinecarboxamide or a pharmaceutically acceptable salt thereof.

45. The pharmaceutical composition according to claim 37 wherein said composition comprises Compound 1 and Carboplatin.

46. The pharmaceutical composition according to claim 37 wherein said composition comprises Compound 1 and Doxorubicin.
